(19) United States
(12) Patent Application Publication
(10) Pub. No.: US 2023/0414603 A1
(43) Pub. Date: Dec. 28, 2023

(54) TREATING OBSESSIVE-COMPULSIVE DISPORDER USING BOL-148

(71) Applicant: CERUVIA LIFESCIENCES LLC, Greenwich, CT (US)

(72) Inventor: Carey Turnbull, Greenwich, CT (US)

(73) Assignee: CERUVIA LIFESCIENCES LLC, Greenwich, CT (US)

(21) Appl. No.: 18/035,342

(22) PCT Filed: Nov. 23, 2021
(86) PCT No.: PCT/US21/60460

§ 371 Date: May 4, 2023

Related U.S. Application Data
(60) Provisional application No. 63/118,025, filed on Nov. 25, 2020.

(51) Int. Cl. A61K 31/48

(52) U.S. Cl. CPC A61K 31/48

(57) ABSTRACT

This invention provides a method for treating a subject afflicted with obsessive-compulsive disorder (OCD) comprising administering to the subject a therapeutically effective amount of a BOL-148-based compound.

### PRIORITY CLAIM

This application claims the benefit of U.S. Provisional Application No. 63/118,025, filed Nov. 25, 2020, the contents of which are incorporated herein by reference.

### FIELD OF THE INVENTION

The present invention relates to compositions and methods for treating obsessive-compulsive disorder (OCD). More specifically, the present invention provides methods of treating OCD with 2-bromo-lysergic acid diethylamide (BOL-148).

### BACKGROUND OF THE INVENTION

Obsessive-compulsive disorder (OCD) is a chronic debilitating disorder wherein an individual has recurring thoughts and behaviors that they feel the need to repeat over and over again. These recurring thoughts and behaviors interfere with the individual's daily life. Obsessions can include repeated thoughts about contamination, fear of losing control, repeated doubts, a need to have things in a particular order, superstitions, aggressive or horrific impulses, and sexual or violent imagery. Individuals attempt to suppress these obsessive thoughts with compulsions such as repetitive behaviors (e.g., hand washing; cleaning; ordering things; checking on things such as switches, locks, and appliances; and checking in on loved ones to make sure they are safe), or mental or physical acts (e.g., praying, counting, tapping, or repeating words).

There are several categories into which OCD sufferers are usually placed. Washers are obsessed with contamination and have hand washing and cleaning compulsions. Checkers are obsessed with checking things associated with harm or danger (e.g., locks and turning off the oven). Counters and arrangers are obsessed with order and symmetry (e.g., numbers, colors, and arrangements). OCD can be difficult to diagnose because these symptoms can also be present with depression, schizophrenia, or other disorders.

OCD affects individuals of all ages from preschool to adulthood, and can be partly genetic. Research suggests that OCD is caused by problems in communication between the front brain and deeper brain structures.

OCD is usually treated with cognitive behavior therapy and/or medication. Exposure and Response Prevention is one therapy used wherein, in the presence of a therapist, the individual is gradually exposed to the thoughts, images, objects, and situations that make them anxious, and the individual makes a choice not to perform a compulsive behavior when the anxiety has been triggered. Over time, this leads to a drop in the individual's anxiety level. Drawbacks to this therapy are that access to appropriately trained therapists is limited, and effective therapy requires a degree of insight and effort and an ability to tolerate symptom discomfort that is difficult for many patients to muster. OCD is also one of the few psychiatric disorders in which invasive brain surgery is an accepted therapeutic option, which is a testament to the profound suffering it can produce.

It has been known for many years that a specific neural circuitry is pathologically affected in many individuals with OCD. Neuroimaging studies have identified hyperactivity in basal ganglia, the amygdala, and key frontal cortical structures (including the orbitofrontal cortex (OFC), medial prefrontal cortex (mPFC), and anterior cingulate cortex (ACC)) in individuals with OCD compared to healthy controls. Furthermore, a number of studies have shown that this hyperactivity correlates with the degree of symptomatology and improves with successful psychotherapeutic or pharmacological treatment, supporting a causal role in pathophysiology. This strong association between regional brain hyperactivity and symptomatology has led to ongoing interest in invasive treatments targeting this circuitry as last-resort interventions in the most severe cases.

Moreover, this hyperactive circuitry can be conceptually linked to the phenomenology of OCD. The cortico-basal ganglia circuitry, especially that linking sensorimotor cortices and the dorsal putamen, has been associated with the formation of inflexible sensory-response behavioral patterns—i.e., habits. A bias towards habit learning has been described in OCD, and dysregulating automated cognitive and behavioral processes may be key to the disorder. In addition, neuroimaging studies have shown greater activation of a network of the brain regions associated with internally-focused thought and valuation (namely, the ventromedial PFC (vmPFC), parahippocampus, middle temporal cortex, and amygdala) in individuals with OCD compared to healthy controls. This greater activation may contribute to the greater subjective experience of doubt that characterizes the disorder. This hyperactive circuitry is also observed in neuroimaging studies employing symptom-provoking tasks, which entail visually presenting OCD-relevant stimuli (e.g., pictures of hand washing) to elicit OCD symptoms, and normalization of circuitry with successful treatment. There is thus a striking conceptual convergence between the functional anatomy that is abnormal in OCD patients (and whose activity is correlated with symptomatology) and current thinking as to the normal function of these circuits.

### Serotonin Reuptake Inhibitors

Serotonin reuptake inhibitors (SR1s) can be used to treat OCD, and are usually administered in high doses. SRIs and their doses include, for example, fluvoxamine (up to 300 mg/day), fluoxetine (40-80 mg/day), sertraline (up to 200 mg/day), paroxetine (40-60 mg/day), citalopram (up to 40 mg/day), clomipramine (up to 250 mg/day), escitalopram (up to 40 mg/day), and venlafaxine (up to 375 mg/day). Drawbacks with such medications include difficulty finding an effective medication or combination of medications, a long lag time (e.g., 3 to 8 weeks) before symptoms improve, and the risk of suicidal thoughts or behavior. 30-40% of patients do not respond at all to SRI treatment and most patients who do respond continue to experience problematic residual symptoms.

### BOL-148

BOL-148 is a derivative of lysergic acid diethylamide (LSD). It is commercially available and can be produced via known methods. While BOL-148 is chemically related to LSD, it is not active as a psychedelic. BOL-148 has previously been used in treating cluster headaches (Halpern, et al.).

There remains a need for more effective and faster-acting treatments for OCD.

### SUMMARY OF THE INVENTION

This invention provides a method for treating a subject afflicted with obsessive-compulsive disorder (OCD) comprising administering to the subject a therapeutically effective amount of a BOL-148-based compound.

### DETAILED DESCRIPTION OF THE INVENTION

### Definitions

In this application, certain terms are used which shall have the meanings set forth as follows.

As used herein, “administer”, with respect to a BOL-148-based compound, means to deliver the compound to a subject's body via any known method suitable for that purpose. Specific modes of administration include, without limitation, oral administration and parenteral administration. Specifically envisioned in the present invention are, without limitation, oral administration, intravenous administration, subcutaneous administration, intra-arterial administration, intramuscular administration, intraperitoneal administration, intranasal administration, intrathecal administration, infusion, and administration via implant.

As used herein, “BOL-148” means 2-bromo-lysergic acid diethylamide (also referred to as 2-bromo-LSD, (8β)-2-bromo-9,10-didehydro-N,N-diethyl-6-methylergoline-8-carboxamide, and CAS RN 478-84-2).

As used herein, a “BOL-148-based compound” includes, without limitation, (i) BOL-148, (ii) pharmaceutically acceptable salts of BOL-148, (iii) deuterated forms of BOL-148, and (iv) prodrug forms of BOL-148. Pharmaceutically acceptable salts of BOL-148 include, without limitation, (i) anionic salts such as chloride (i.e., HCl), bromide, iodide, sulfate, nitrate, phosphate, citrate, methanesulfonate, trifluoroacetate, and acetate salts; (ii) cationic salts such as sodium, potassium, magnesium, calcium, and ammonium (e.g., tetramethylammonium) salts; (iii) acid salts such as acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, and undecanoate salts; and (iv) basic salts such as alkali metal (e.g., sodium), alkaline earth metal (e.g., magnesium), ammonium, and N-(alkyl)₄ salts. Prodrug forms of BOL-148 include, without limitation, esters. Deuterated forms of BOL-148, include, without limitation, BOL-148 containing a single hydrogen-deuterium replacement, BOL-148 containing two hydrogen-deuterium replacements, BOL-148 containing three hydrogen-deuterium replacements, and BOL-148 having all of its hydrogen atoms replaced by deuterium.

The following additional embodiments of BOL-148-based compounds, based on the numbered BOL-148 structure below, are exemplary.

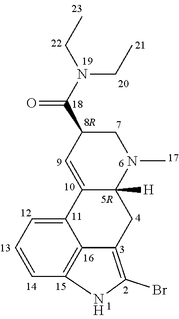 [SMILES: CCN(CC)C(=O)[C@@H]1C=C2c3cccc4[nH]c(Br)c(c34)C[C@H]2N(C)C1]

In a first embodiment, this invention provides BOL-148 containing a single deuterium replacement of a hydrogen atom bound to one of the following atoms: (i) C-4; (ii) C-5 (i.e., 5R); (iii) C-7; (iv) C-8; (v) C-12; (vi) C-13; (vii) C-14; (viii) C-17; (ix) C-20; (x) C22; (xi) C21; or (xii) C23. In a second embodiment, this invention provides BOL-148 containing two deuterium replacements of two hydrogen atoms bound to the following atoms: (i) C-20 and C22; or (ii) C21 and C23. In a third embodiment, this invention provides BOL-148 containing two deuterium replacements of two hydrogen atoms bound to one of the following atoms: (i) C-4; (ii) C-7; (iii) C-17; (iv) C-20; (v) C22; (vi) C21; or (vii) C23. In a fourth embodiment, this invention provides BOL-148 containing three deuterium replacements of three hydrogen atoms bound to one of the following atoms: (i) C-17; (ii) C21; or (iii) C23.

In this invention, the BOL-148-based compound (e.g., BOL-148) is preferably formulated using one or more routinely used pharmaceutically acceptable carriers. Such carriers are well known to those skilled in the art.

For example, oral drug delivery systems include tablets (e.g., a compressed preparation containing (i) 5-10% of the BOL-148-based compound, (ii) 80% of fillers, disintergrants, lubricants, glidants, and binders, and (iii) 10% of compounds which ensure easy disintegration, disaggregation, and dissolution of the tablet in the stomach or the intestine). Tablet dissolution time can be modified for a rapid effect or for sustained release. Special coatings can make the tablet resistant to stomach acid such that it only disintegrates in the duodenum, jejunum, and colon as a result of enzyme action or alkaline pH. Tablets can be coated with sugar, varnish, or wax to mask the drug's unpleasant taste. Oral drug delivery systems also include capsules (e.g., having a gelatinous envelope enclosing the BOL-148-based compound). Capsules can be designed to remain intact for some hours after ingestion to delay absorption, and may also contain both slow and fast release particles to produce rapid and sustained absorption in the same dose.

Injectable forms (e.g., solutions, suspensions, and emulsions) include, without limitation, sterile aqueous solutions or dispersions and sterile powders for reconstitution into sterile injectable solutions or dispersions. The carrier can be, for examples, a solvent or dispersing medium containing, for example, water, ethanol, a polyol (e.g., glycerol, propylene glycol, liquid polyethylene glycol, and the like), suitable mixtures thereof, and vegetable oils. Proper fluidity can be maintained, for example, by the use of a coating such as lecithin, by the maintenance of the required particle size in the case of dispersion and by the use of surfactants. Nonaqueous vehicles such as cottonseed oil, sesame oil, olive oil, soybean oil, corn oil, sunflower oil, or peanut oil and esters, such as isopropyl myristate, may also be used as solvent systems for compound compositions. Additionally, various additives that enhance the stability, sterility, and isotonicity of the compositions, including antimicrobial preservatives, antioxidants, chelating agents, and buffers, can be added. Prevention of microorganism action can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents for delaying absorption, for example, aluminum monostearate and gelatin.

Injectable forms can be administered parenterally in the form of slow-release subcutaneous implants or targeted delivery systems employing, for example, monoclonal antibodies, vectored delivery, polymer matrices, liposomes, and microspheres. Examples of delivery systems useful in the present invention include those disclosed in U.S. Pat. No. 5,225,182; 5,169,383; 5,167,616; 4,959,217; 4,925,678; 4,487,603; 4,486,194; 4,447,233; 4,447,224; and 4,439,196. Other such implants, delivery systems, and modules are well known to those skilled in the art.

As used herein, a “human subject” can be of any age, gender, or state of co-morbidity. In one embodiment, the subject is male, and in another, the subject is female. In another embodiment, the subject is co-morbid (e.g., afflicted with both OCD and another disorder such as major depressive disorder (MDD), tics (e.g., in male subjects younger than 30 years old), or panic disorder (PD) (e.g., in female subjects at least 60 years old)). In a further embodiment, the subject is not co-morbid. In still another embodiment, the subject is younger than 20 years old, younger than 25 years old, younger than 30 years old, younger than 35 years old, younger than 40 years old, younger than 45 years old, younger than 50 years old, younger than 55 years old, or younger than 60 years old. In yet another embodiment, the subject is at least 60 years old, at least 65 years old, at least 70 years old, at least 75 years old, at least 80 years old, at least 85 years old, or at least 90 years old. In a further embodiment, the subject is refractory to treatment with one or more drugs. In one example, the subject is refractory to treatment with one or more SRIs such as fluvoxamine, fluoxetine, sertraline, paroxetine, citalopram, clomipramine, escitalopram, or venlafaxine.

As used herein, the term “OCD symptom” includes, without limitation, (i) fear of contamination or dirt; (ii) doubting and having difficulty tolerating uncertainty; (iii) needing things to be orderly and symmetrical; (iv) aggressive or horrific thoughts about losing control and harming oneself or others; (v) unwanted thoughts (e.g., about aggression, or sexual or religious subjects); (vi) other obsessive thoughts or behaviors; and (vii) other compulsive thoughts or behaviors.

As used herein, “reducing the likelihood” of recurrence of an OCD symptom includes, without limitation, reducing such likelihood by at least 10%, at least 20%, at least 30%, at least 40%, at least 50%, at least 60%, at least 70%, at least 80%, at least 90%, or at least 99%.

As used herein, the term “subject” includes, without limitation, a mammal such as a human, a non-human primate, a dog, a cat, a horse, a sheep, a goat, a cow, a rabbit, a pig, a hamster, a rat, and a mouse. The present methods are envisioned for these non-human subjects, mutatis mutandis, as they are for human subjects in this invention.

As used herein, a “therapeutically effective amount” of a BOL-148-based compound (e.g., BOL-148) includes, without limitation, (i) 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.05 mg, 2.10 mg, 2.15 mg, 2.20 mg, 2.25 mg, 2.30 mg, 2.35 mg, 2.40 mg, 2.45 mg, 2.50 mg, 2.6 mg, 2.7 mg, 2.8 mg, 2.9 mg, 3.0 mg, 4.0 mg, 5.0 mg, 6.0 mg, 7.0 mg, 8.0 mg, 9.0 mg, 10 mg, 15 mg, 20 mg, 25 mg, 30 mg, 35 mg, 40 mg, 45 mg, or 50 mg; (ii) 0.1 mg to 0.5 mg, 0.5 mg to 1.0 mg, 1.0 mg to 1.5 mg, 1.5 mg to 2.0 mg, 2.0 mg to 2.10 mg, 2.10 mg to 2.20 mg, 2.10 mg to 2.30 mg, 2.10 mg to 2.40 mg, 2.20 mg to 2.30 mg, 2.20 mg to 2.40 mg, 2.30 mg to 2.40 mg, 2.40 mg to 2.50 mg, 2.50 mg to 3.0 mg, 3.0 mg to 5.0 mg, 5.0 mg to 10 mg, or 10 mg to 50 mg; (iii) 1 μg/kg, 5 μg/kg, 10 μg/kg, 15 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 31 μg/kg, 32 μg/kg, 33 μg/kg, 34 μg/kg, 35 μg/kg, 36 μg/kg, 37 μg/kg, 38 μg/kg, 39 μg/kg, 40 μg/kg, 45 μg/kg, 50 μg/kg, 60 μg/kg, 70 μg/kg, 80 μg/kg, 90 μg/kg, 100 μg/kg, 200 μg/kg, 300 μg/kg, 400 μg/kg, 500 μg/kg, 600 μg/kg, 700 μg/kg, 800 μg/kg, 900 μg/kg, or 1,000 μg/kg; or (iv) 1 μg/kg to 5 μg/kg, 5 μg/kg to 10 μg/kg, 10 μg/kg to 15 μg/kg, 15 μg/kg to 20 μg/kg, 20 μg/kg to 25 μg/kg, 25 μg/kg to 30 μg/kg, 25 μg/kg to 35 μg/kg, 30 μg/kg to 35 μg/kg, 35 μg/kg to 40 μg/kg, 40 μg/kg to 50 μg/kg, 50 μg/kg to 100 μg/kg, 100 μg/kg to 500 μg/kg, or 500 μg/kg to 1,000 μg/kg.

As used herein, “treating” a subject afflicted with OCD means ameliorating at least one OCD symptom, a plurality of OCD symptoms, or all OCD symptoms in the subject. Examples of treating a subject afflicted with OCD include, without limitation, (i) slowing, stopping, or reversing the progression of one or more OCD symptoms, and/or (ii) reducing or eliminating the likelihood of the symptoms' recurrence. In a preferred embodiment, treating a subject afflicted with OCD includes (i) reversing the progression of at least one OCD symptom, a plurality of OCD symptoms, or all OCD symptoms, and/or (ii) reducing or eliminating the likelihood of recurrence of at least one OCD symptom, a plurality of OCD symptoms, or all OCD symptoms. In another preferred embodiment, treating a subject afflicted with OCD includes lowering the subject's score on the Yale-Brown Obsessive-Compulsive Scale (Y-BOCS; see, e.g., Goodman, et al., Arch Gen Psychiatry, 46 (11): 1006-1011 (1989)) by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. Ideally, in the present method, treating a subject afflicted with OCD includes lowering the subject's Y-BOCS score by at least 35%.

### Embodiments of the Invention

This invention provides a method for treating a subject afflicted with obsessive-compulsive disorder (OCD) comprising administering to the subject a therapeutically effective amount of a BOL-148-based compound.

In a preferred embodiment of the present method, the subject is a human subject. In another preferred embodiment of the present method, the BOL-148-based compound is BOL-148. In a further preferred embodiment of the present method, the administering is oral.

In one preferred embodiment of the present method, the method comprises administering to the subject three doses of the BOL-148-based compound, wherein (i) the doses are administered five days apart, and (ii) each dose is less than 50 μg/kg. In another preferred embodiment of the present method, the method comprises administering to the subject three doses of the BOL-148-based compound, wherein (i) the doses are administered five days apart, and (ii) each dose is 30 μg/kg.

In one embodiment, the therapeutically effective amount of BOL-148-based compound is administered as a single, one-time-only dose. In another embodiment, the therapeutically effective amount of BOL-148-based compound is administered as a plurality of doses over a period of days, weeks, or months. For example, in a preferred embodiment, the therapeutically effective amount of BOL-148-based compound (e.g., 25 μg/kg, 30 μg/kg, or 35 μg/kg of BOL-148 dissolved in distilled water) is administered a plurality of times (e.g., two times, three times, four times, or five times), wherein each dose is administered several days apart (e.g., four days apart, five days apart, or six days apart).

The following additional embodiments of the present method are exemplary.

In a first embodiment, this invention provides a method for treating a human subject afflicted with OCD comprising orally administering to the subject three doses of BOL-148 (e.g., in tablet form, capsule form, or dissolved in distilled water), wherein (i) the doses are administered five days apart, and (ii) each dose is less than 50 μg/kg.

In a second embodiment, this invention provides a method for treating a human subject afflicted with OCD comprising orally administering to the subject three doses of BOL-148 (e.g., in tablet form, capsule form, or dissolved in distilled water), wherein (i) the doses are administered five days apart, and (ii) each dose is 35 μg/kg.

In a third embodiment, this invention provides a method for treating a human subject afflicted with OCD comprising orally administering to the subject three doses of BOL-148 (e.g., in tablet form, capsule form, or dissolved in distilled water), wherein (i) the doses are administered five days apart, and (ii) each dose is 30 μg/kg.

In a fourth embodiment, this invention provides a method for treating a human subject afflicted with OCD comprising orally administering to the subject three doses of BOL-148 (e.g., in tablet form, capsule form, or dissolved in distilled water), wherein (i) the doses are administered five days apart, and (ii) each dose is 25 μg/kg.

The present methods are envisioned for BOL-148-based compounds other than BOL-148, mutatis mutandis, as they are for BOL-148 in this invention.

In the present therapeutic method, one or more OCD symptom types are treated. These symptom types include, without limitation, (i) fear of contamination or dirt; (ii) doubting and having difficulty tolerating uncertainty; (iii) needing things to be orderly and symmetrical; (iv) aggressive or horrific thoughts about losing control and harming oneself or others; and (v) unwanted thoughts (e.g., about aggression, or sexual or religious subjects). In one embodiment of the present therapeutic method, the method ameliorates symptom type (i). In another embodiment, the method ameliorates symptom type (ii). In a further embodiment, the method ameliorates symptom type (iii). In a further embodiment, the method ameliorates symptom type (iv). In yet a further embodiment, the method ameliorates symptom type (v). Preferably, the present method ameliorates all OCD symptoms in the subject. In a preferred embodiment, the present therapeutic method is performed on a subject in conjunction with psychotherapy.

This invention also provides a composition suitable for treating OCD comprising a therapeutically effective amount of a BOL-148-based compound (e.g., BOL-148) and a pharmaceutically effective carrier. Preferably, the present composition is formulated at one of the doses exemplified herein using one of the pharmaceutically acceptable carriers exemplified herein.

Throughout this application, various publications are referenced. The disclosures of these publications in their entireties are hereby incorporated by reference into this application in order to more fully describe the state of the art to which this invention pertains.

### REFERENCES

DeCant, et al., U.S. Pat. No. 4,447,224.
Ferrara, U.S. Pat. No. 4,486,194.
Goodman, et al., Arch Gen Psychiatry, 46 (11): 1006-1011 (1989).
Gyory, et al., U.S. Pat. No. 5,169,383.
Haak, et al., U.S. Pat. No. 5,167,616.
Halpern, et al., U.S. Pat. No. 8,415,371.
Harris, U.S. Pat. No. 4,487,603.
Higuchi, U.S. Pat. No. 4,439,196.
Karst, et al., Cephalalgia, 2010 30(9):1140-1144.
Mayfield, U.S. Pat. No. 4,447,233.
Ranney, U.S. Pat. No. 4,925,678.
Sanders and Domb, U.S. Pat. No. 4,959,217.
Sewell, U.S. Pat. No. 8,859,579.
Sharma, U.S. Pat. No. 5,225,182.

What is claimed is:

1. A method for treating a subject afflicted with obsessive-compulsive disorder (OCD) comprising administering to the subject a therapeutically effective amount of a BOL-148-based compound.

2. The method of claim 1, wherein the subject is a human subject.

3. The method of claim 1 or 2, wherein the BOL-148-based compound is BOL-148.

4. The method of any of claims 1-3, wherein the administering is oral.

5. The method of any of claims 1-4, wherein the method comprises administering to the subject three doses of the BOL-148-based compound, wherein (i) the doses are administered five days apart, and (ii) each dose is less than 50 μg/kg.

6. The method of any of claims 1-5, wherein the method comprises administering to the subject three doses of the BOL-148-based compound, wherein (i) the doses are administered five days apart, and (ii) each dose is 30 μg/kg.

7. The method of any of claims 1-6, wherein the subject is undergoing psychotherapy.
